(19) United States
(12) Patent Application Publication
(10) Pub. No.: US 2010/0228082 A1
(43) Pub. Date: Sep. 9, 2010

(54) DEVICE, SYSTEM AND METHOD FOR INTERACTING WITH A CELL OR TISSUE IN A BODY

(75) Inventors: Hans Zou, Windsor, NJ (US); Stein Kuiper, Vught (NL); Bernardus Hendrikus Wilhelmus Hendriks, Eindhoven (NL); Jan Frederik Suijver, Dommelen (NL); Anke Pierik, Eindhoven (NL); Judith Margreet Rensen, Eindhoven (NL); Johan Frederik Dijksman, Weert (NL); Jeff Shimizu, Cortlandt Manor, NY (US); Jacob Marinus Jan Den Toonder, Helmond (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/376,460

(22) PCT Filed: Apr. 12, 2007
(86) PCT No.: PCT/IB2007/051326

§ 371 Date: Feb. 5, 2009

(51) Int. Cl. A61B 1/00

(52) U.S. Cl. 600/104

(57) ABSTRACT

A device or support, system and method are provided for precisely targeted and/or highly controlled interaction with a targeted cell or tissue in a body, with the support providing an ergonomic connecting interface for selectively connecting the support to a cell or tissue with at least minimal adverse affect to such cell or tissue, the system providing a remote facility that may be operatively associated with the support, and the method providing steps for employing the support and/or system so as to, inter alia, improve treatment, diagnostic and/or monitoring techniques and increase sensitivity and specificity with respect to interacting with, for example, diseased or abnormal cells or tissue without adversely effecting surrounding healthy cells or tissue, and/or the body in general.

The present disclosure is related to U.S. Provisional Patent Application No. 60/644,540, entitled “Electronically Controlled Capsule For Releasing Radiation”, and filed Jan. 18, 2005, U.S. Provisional Patent Application No. 60/644,539, entitled “Electronically Controlled Capsule”, and filed Jan. 18, 2005, U.S. Provisional Patent Application No. 60/644,538, entitled “Electronically Controlled Ingestible Capsule”, and filed Jan. 18, 2005, U.S. Provisional Patent Application No. 60/644,518, entitled “System And Method For Controlling Traversal Of An Ingested Capsule”, and filed Jan. 18, 2005, U.S. Provisional Patent Application No. 60/606,276, entitled “Electronically Controlled Pill And System For Delivering At Least One Medicament”, and filed Sep. 1, 2004, U.S. Provisional Patent Application No. 60/605,364, entitled “Electronically And Remotely Controlled Pill And System For Delivering At Least One Medicament”, and filed Aug. 27, 2004, U.S. Provisional Patent Application No. 60/738,238, entitled “System and Method for Interacting With a Cell or Tissue”, and filed Nov. 18, 2005, U.S. Provisional Patent Application No. 60/805,223, entitled “Electronic Capsule And Method For Treating Gastrointestinal Disease”, and filed Jun. 20, 2006, and U.S. Provisional Patent Application No. 60/805,645, entitled “Medicament Delivery System And Process”, and filed Jun. 23, 2006, with each of the foregoing references being assigned to the Assignee of the present disclosure and hereby being expressly incorporated by reference as part hereof.

The present disclosure is directed generally to interacting with targeted cells and/or tissues in a body, and more particularly to a device and method for obtaining, inter alia, quality observation information, improved treatment precision, and enhanced diagnostic/treatment for results precisely controlled substance or particle (e.g., medicament, nanoparticles, etc.) delivery, as well as accurate and consistent treatment or diagnostic processes.

Devices having imaging or observation capabilities are known in the art. For example, U.S. Pat. No. 6,240,312 to Alfano et al., issued May 29, 2001, which patent is hereby incorporated herein by reference, reads on an ingestible capsule having a camera that acquires diagnostic images as it traverses the alimentary tract. In addition, U.S. Pat. No. 6,324,418 to Crowley et al., issued Nov. 27, 2001, which patent is also hereby incorporated herein by reference, reads, at least in part, on a capsule for performing tissue spectroscopy.

Notwithstanding the benefits provided via the imaging and diagnosing capabilities associated with the above-noted devices and the like, there remains a need for a device and/or system suitable for, among other things, keeping the interaction between a diagnostic and/or treatment tool or support (e.g., an endoscope or capsule) and a cell or tissue in a body as static as possible during an observation or treatment process. For example, when attempting to record pathology using, for instance, a sensing probe provided on the tip of an endoscope, movement of the endoscope and/or cell or tissue under investigation relative to each other can result in blurred images and missing key data.

Additionally, there is a further need for a device and/or system that is suitable for, among other things, accomplishing precisely targeted and/or highly controlled substance or particle delivery. That is, although devices have been developed and are known (e.g., capsules) that are adequate for certain medicament delivery applications (see, e.g., U.S. Pat. Nos. 5,951,538 and 6,719,684), typically these devices have relatively inflexible and substantially limited capabilities and/or utilizations. For example, such devices typically have the ability to support medicament release or delivery only haphazardly and/or only for a finite and undeterminable duration. In addition, such known devices may also have an adverse effect or interactions with a body (e.g., cause significant bleeding) and/or the functions or processes (e.g. fluid flow, digestion, etc.) thereof. Hence, there is a need for a device or a support and/or system that can, for example and without limitation: (i) improve medicament efficacy, (ii) reduce or eliminate causes relating to patient non-compliance to prescriptions, (iii) help maintain a steady medicament concentration, (iv) help reduce or minimize needed medicament administrations, diagnostic procedures, or other similar treatments/monitoring needed, (v) monitor a disease or a cell or tissue in real-time over a treatment course, and/or (vi) stabilize an interaction between a diagnostic and/or treatment tool or support and a cell or tissue.

With the foregoing in view, there is, according to a beneficial aspect of present disclosure, an exemplary support discussed for which at least one ergonomic connecting interface capable of selectively connecting the support to a cell or tissue in a body is provided. The exemplary support, as disclosed, has at least one module that is capable of accomplishing one or more operations relative to a cell or tissue.

According to another beneficial aspect of the present disclosure, a module may include one or more reservoirs capable of retaining at least one substance or solution, such substance or solution possibly including a medicament, nano-particles, or other cell or tissue interactive material. Preferably, at least one delivery mechanism may be operatively associated with a reservoir, such delivery mechanism preferably being capable of selectively delivering at least one substance or solution. In accordance with another aspect, a module may include one or more imagers capable of imaging a cell or tissue, and/or one or more sensors capable of detecting one or more characteristics or pathologies directly or indirectly associated with a cell or tissue, and/or one or more illuminators capable of providing light to or about a cell or tissue.

According to an advantageous aspect of the present disclosure, a support may have a controller operatively associated with at least one module so as to allow for the controlled operation thereof. Such a controller may, according to another advantageous aspect of the present disclosure, include a communicator capable of communicating with one or more remote facilities.

In a further beneficial aspect of the present disclosure, a controller can be operatively associated with a delivery mechanism so as to allow for the controlled delivery of one or more substances. Such a controller may include one or more sensors operative, at least in part, to signal, for instance, the controlled delivery of the one or more substances. Such a controller may also include a communicator capable of communicating with at least one remote facility, the remote facility preferably having a processing device or system that can signal the controlled delivery of the one or more substances or solutions.

According to still a further beneficial aspect of the present disclosure, an ergonomic connecting interface may include one or more polymer adhesives or bio-adhesives, one or more micro-elements and/or nano-elements, such as, one or more electro-statically actuated micro-structures (e.g., electrostatic actuated elements or hooks), one or more nano-pillars or a nano-pillar array, and/or one or more suction elements or a suction system. The ergonomic connecting interface of the present disclosure may also include further features such as, for example, one or more changeable wetting materials.

In accordance with still another beneficial aspect of the present disclosure, an exemplary system is discussed in which at least one support that is preferably capable of insertion into a body has at least one ergonomic connecting interface operatively associated therewith. Such support also preferably having at least one module operatively associated therewith suitable to accomplish at least one operation relative to a cell or tissue in the body. In a preferred aspect of the exemplary system, such system includes a controller that may, for example, be operatively associated with a module. In another preferred aspect, a module may be operatively associated with a remote facility so as to communicate and/or otherwise interact therewith.

According to yet still another advantageous aspect of the present disclosure, a method is discussed for interacting with a cell or tissue that may include at least the following steps: (i) introducing at least one support to one or more areas in a body, (ii) connecting at least one support to a targeted cell or tissue of the body, (iii) performing, via at least one support, at least one operation relative to the cell or tissue, and (iv) disconnecting the at least one support in response to an event such as, for example, and without limitation, the performance of an operation, or the lapse of a predetermined time amount, or the exhaustion of a predetermined resource amount (e.g., substance or medicament), or the detection of an adverse reaction relative to the cell or tissue, or a signal generated by the support (e.g., via a sensor) or a remote facility (e.g., a monitoring device or processor with means for user input or control).

Additional advantageous features, aspects and/or functions relating to the present disclosure will be apparent from the detailed description which follows, particularly when reviewed together with the appended figures, which figures are referenced to assist those of ordinary skill in the art to which the subject matter of the present disclosure appertains to better understand the illustrative examples of the present disclosure, wherein:

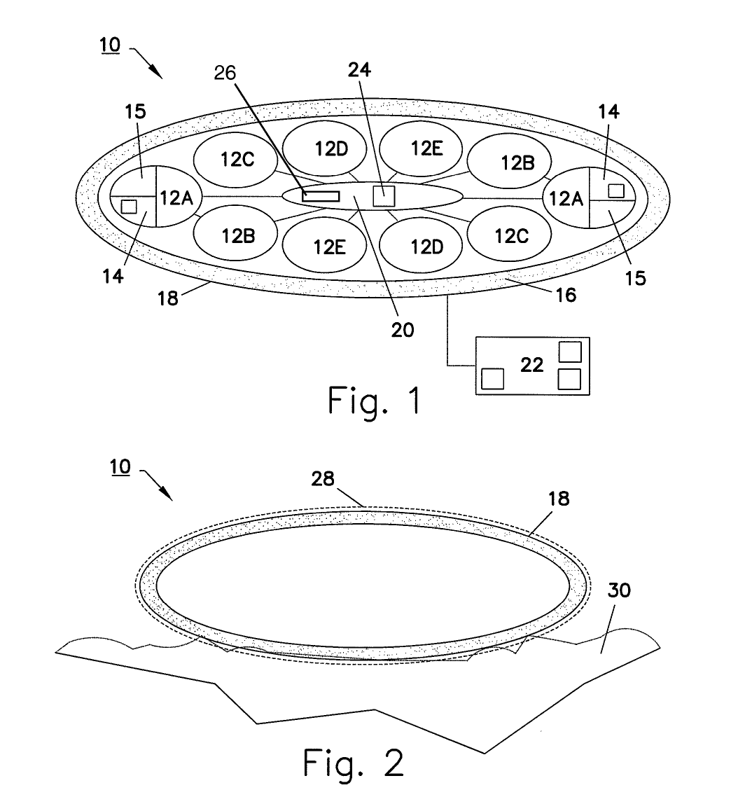

FIG. 1 is a schematic representation of a device or support and a system in accordance with an illustrative aspect of the present disclosure;

FIG. 2 is a schematic representation of a device or support with a connecting interface in accordance with an illustrative aspect of the present disclosure;

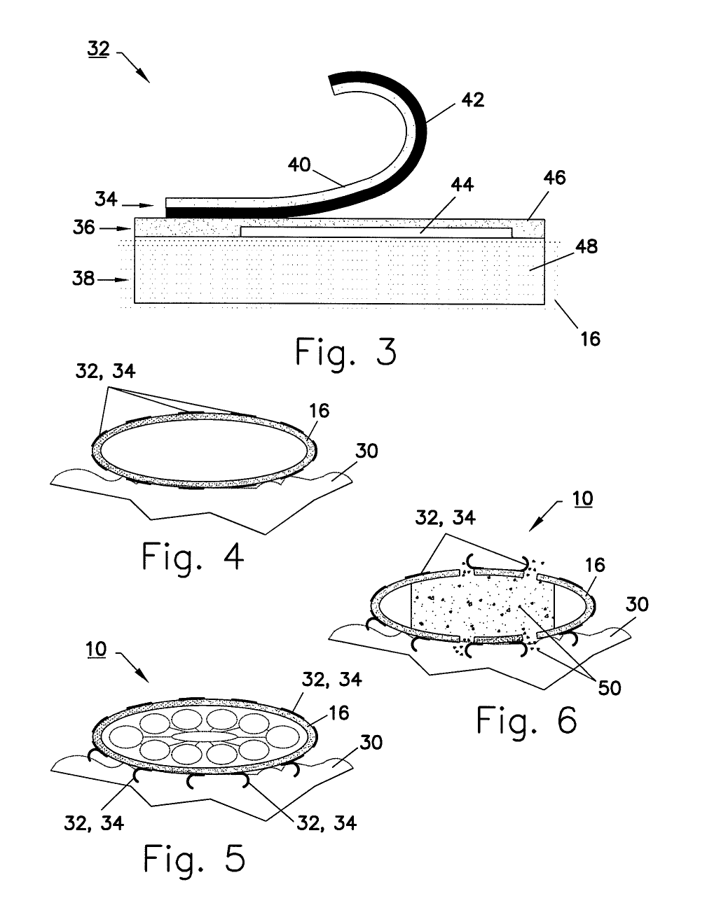

FIG. 3 is a schematic representation of an exemplary micro-structure element that can be operatively associated with a connecting interface of a support according to an illustrative aspect of the present disclosure;

FIG. 4 is a schematic representation of a support with a connecting interface according to another illustrative aspect of the present disclosure;

FIG. 5 is another schematic representation of the exemplary connecting interface of FIG. 4;

FIG. 6 is yet another schematic representation of the exemplary connecting interface of FIG. 4;

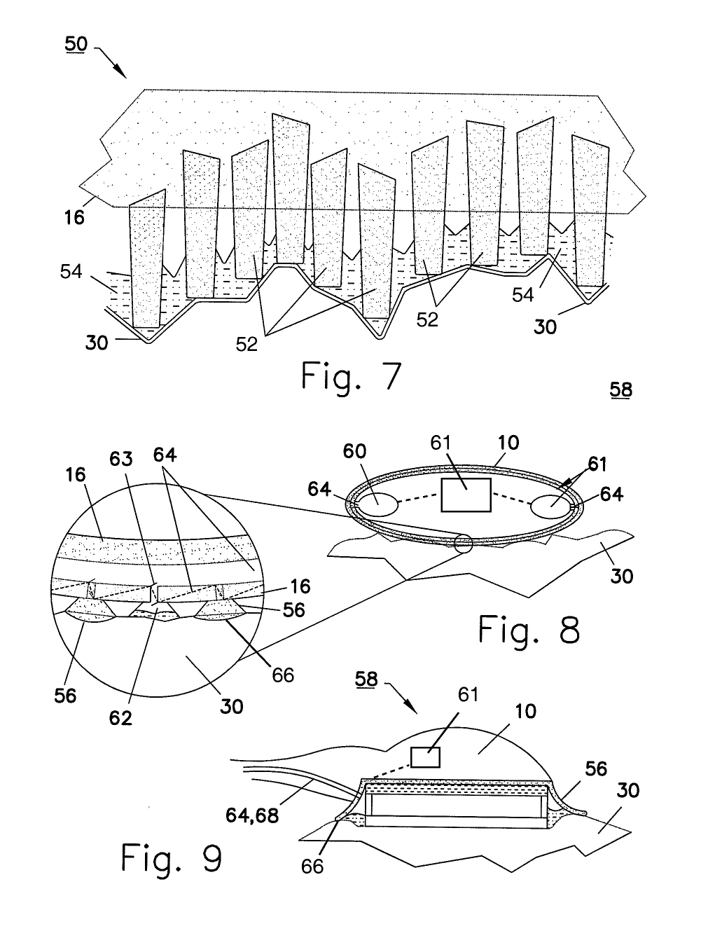

FIG. 7 is a schematic representation of an exemplary nano-pillar array that can be operatively associated with a connecting interface of a support in accordance with still another illustrative aspect of the present disclosure;

FIG. 8 is a schematic representation of an exemplary suction system that can be operatively associated with a connecting interface of a support according to a further illustrative aspect of the present disclosure;

FIG. 9 is a schematic representation of another exemplary suction system that can be operatively associated with a connecting interface of a support according to yet a further illustrative aspect of the present disclosure; and

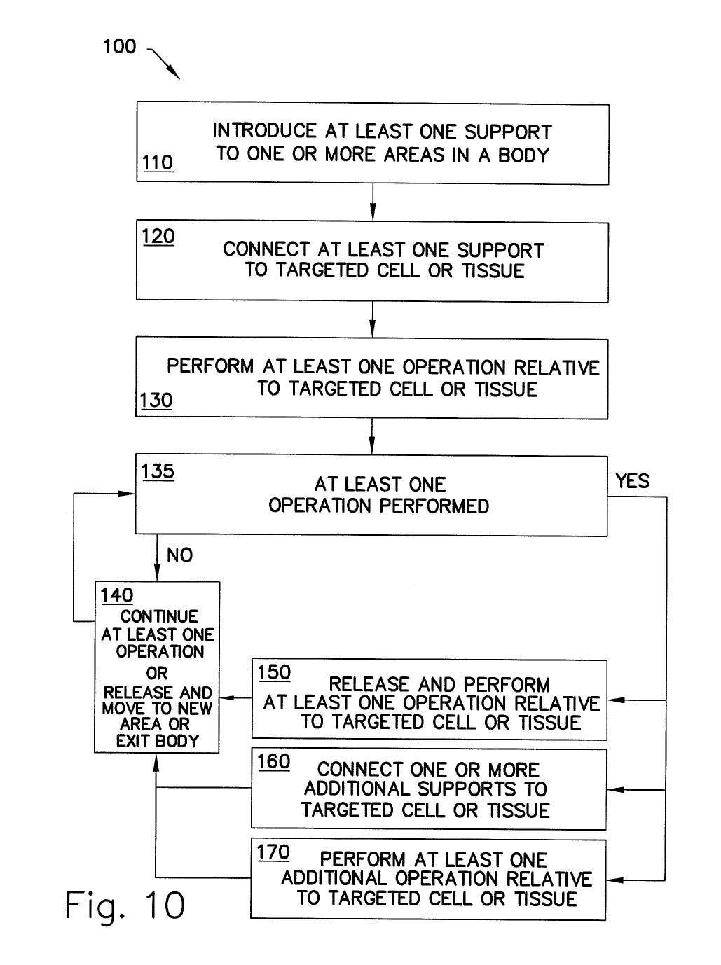

FIG. 10 is a flow diagram of a method according to still a further illustrative aspect of the present disclosure.

With reference to the drawings, it should be understood that notwithstanding the following detailed description of the various examples and/or aspects of the present disclosure referring to the drawings which form a part hereof, other additional and/or alternative examples, aspects and/or features may equally be used without departing from the scope of the present disclosure as the advantageous features of the present disclosure may be employed in any of a variety of applications including, for example, dietary applications such as disclosed and suggested via pending U.S. Provisional Application Ser. No. 60/787,454, filed Mar. 30, 2006, and entitled “Expandable Digestive Pill and Method of Use Thereof”.

With initial reference to FIG. 1, there is shown an exemplary support 10 in accordance with an illustrative aspect of the present disclosure. As shown, such support 10 includes at least one operating module 12a-12e capable of accomplishing one or more operations relative to a cell or tissue.

For example, in an aspect of the present disclosure, the support 10 may have one or more delivery modules 12a for delivering any of a variety of substances (e.g., diagnostic or treatment medicaments, etc.). In a preferred aspect of the present disclosure, a delivery module 12a can be operatively associated with a reservoir module 12b. However, in other aspects of the present disclosure, this need not be the case, as it is foreseeable that in certain situations, the support 10 may itself be suitable to directly accommodate, for example, one or more medicaments (e.g., via surface coating or infusion) and hence may thereby serve, at least in part, as both a reservoir and a delivery means. In still other aspects of the present disclosure, at least one reservoir module 12b may be accommodated or defined within the support 10, each reservoir module 12b having a delivery module 12a, such as, for example, a valve and/or a pump, operatively associated therewith. In a further aspect of the present disclosure, each delivery module 12a may be operatively associated with a control module 14 and/or a sensor 15 so as to be directly or indirectly controlled thereby (e.g., via control signals) such that the contents of a reservoir may be released and/or delivered, as desired, to a targeted cell or tissue. Such a release or delivery may be accomplished in accordance with a prescribed or programmable pattern necessary to address a particular disease or medicament parameter. Thus, the support 10 of the present disclosure may beneficially be utilized for a variety of diagnostic and/or treatment functions.

It is noted that those skilled in the pertinent art, from the present disclosure, will readily appreciate variations to the foregoing delivery/reservoir arrangement without departing from the spirit and/or scope of the present disclosure. For example, an artificial muscle formed of a polymer that controllably expands or contracts in response to an applied electrical signal so as to apply controlled pressure to the reservoir and/or the contents thereof and thereby cause an effect (e.g., mixing, dispensing, etc.) with respect to the contents as desired may be utilized.

Other modules that may be operatively associated with the support 10 according to the present disclosure include, without limitation, (i) an illuminating module 12c (e.g., an LED or other suitable light source) suitable for emitting at least one light or laser beam that impacts and is reflected from a targeted cell or tissue near the support 10 so that the distinct light reflectivity properties associated with the targeted cell or tissue are identifiable and/or measurable; (ii) a detecting module 12d (e.g., a sensor array, CCD or the like) suitable for sensing incident light and generating a corresponding signal so as to, for example, facilitate distinguishing a targeted cell or tissue from adjacent cells or tissues; and/or (iii) at least one imaging module 12e (e.g., a micro-video CCD) suitable for collecting and/or transmitting images, so that a person or a computer-aided detection system (e.g., CAD, CADx, etc.) may detect changes in the texture of a targeted cell or tissue.

With continued reference to FIG. 1, the support 10, according to an illustrative aspect of the present disclosure, can be made from bio-compatibles materials such that the support 10 is biocompatible for at least the amount of time that it remains in a body. For instance, in an aspect of the present disclosure, the support 10 may be made from materials typically used to fabricate implantable devices (e.g., pacemaker leads and cardiac prosthesis devices). Suitable materials might include, for example, Pellethane® 2363 polyether urethane series of materials available from Dow Chemical Company, and Elasthane polyether urethane available from the Polymer Technology Group, Inc. Other exemplary materials that might also be appropriate include PurSil® and CarboSil® also available from the Polymer Technology Group, Inc.

The support 10, in different aspects of the present disclosure, can have any of a variety of shapes, sizes, colors, textures and/or other characteristics or properties necessary to accomplish any of a variety of different ergonomic and/or functional purposes consistent with the present disclosure. For example, according to a preferred aspect of the present disclosure, the support 10 includes at least one ergonomic connecting interface 16, or a connecting interface that accounts for the interaction between the support 10 and the body so as to preferably optimize body comfort and well being, as well as the performance of the support 10 and/or any feature or functions provided thereby, or in associated therewith. According to another aspect of the present disclosure, the support 10 may include a micro-porous membrane with holes ranging in size from sub-micron to a few microns in diameter. The membrane can be infused or impregnated with a substance or particles, wherein upon stretching of the membrane, such as by some mechanical means, the substance or particles may be released at a controlled rate over a specific area. In an alternative aspect, the substance or particles may be coated onto a surface 18 of the support 10 so as to be deliverable to a specific site. Pressure, heat, laser light, etc., may facilitate transfer of the coated substance or particles from the surface of the support 10 to a targeted area in the body.

It is again noted that those skilled in the art will readily appreciate, from the present disclosure that variations to the support 10 may be made without departing from the spirit and scope of the present disclosure. For example, at least a portion of the support 10 may be substantially transparent or translucent so as to allow light of a desired wavelength to be emitted, via the illuminating module 12e, in at least one direction. According to another example, the support 10 may take the form of an endoscope or like device, which endoscope can have a tip with one or more modules operatively associated therewith.

Still referencing FIG. 1, according to a beneficial aspect of the present disclosure, the support 10 may also have a controller operatively associated therewith. For example, as shown, a controller 20 may be provided so as to be operatively associated with any one or more of the modules of the support 10 in order to influence or control the operation of such module(s). The controller 20 preferably has both processing and communication capabilities suitable to facilitate interaction with the module(s) of the support 10 and/or a remote facility 22 (e.g., a computer), and/or the connecting interface 16. For example, the controller 20 may include a microprocessor 24 suitable to, without limitation, effect various control operations relating to the module(s) of the support 10, and a transmitter/receiver 26 suitable to, without limitation, communicate with corresponding elements of the remote facility 22 (e.g., via wireless signals). It is noted that one skilled in the pertinent art would readily recognize that any of a variety of processing and/or communication techniques may be effectively utilized, and that any such technique should be deemed to be within the scope of the present disclosure.

Having identified and discussed various beneficial aspects and features that may be associated with the device of the present disclosure, with reference now to FIGS. 2-9, various illustrative examples of how such device might be characterized with particular respect to the connecting interface thereof are now discussed.

Referring to FIG. 2, according to one illustrative aspect of the present disclosure, a connecting interface may include one or more adhesives or adhesive layers (e.g., polymer based) 28 associated with a surface 18 of a support 10. In addition, the properties of such adhesive(s) or adhesive layer(s) (indicated as a dashed line) may, in a preferred aspect of the present disclosure, be altered to be bio-adhesive, negatively charged, or positively charged, depending on, for example, the intended targeted cell or tissue. That is, the connection of the support 10 to a targeted cell or tissue 30 may be improved or modified by changing the properties of the adhesive(s) or adhesive layer(s) associated with the support 10 so as to enhance their affinity for a particular residue expressed on a targeted cell or tissue surface or enhance their affinity for a protein or receptor associated with such cell or tissue. A bio-adhesive, for example, can be created by incorporating a bio-adhesive material into the solid hydrophobic support surface medium or matrix and/or the adhesive layer matrix, or by incorporating a bio-adhesive material in a pH-sensitive support surface matrix and/or a pH-sensitive adhesive layer matrix. Other techniques for developing an effective bio-adhesive interface will be readily apparent to those skilled in the pertinent art.

According to a beneficial aspect of the present disclosure, polymers with enhanced bio-adhesive properties can, for example, be provided wherein anhydride monomers or oligomers are incorporated into the polymer. The oligomer excipients can be blended or incorporated into a wide range of hydrophilic and hydrophobic polymers including proteins, polysaccharides and synthetic biocompatible polymers. Anhydride oligomers can be combined with metal oxide particles to improve bio-adhesion in addition to the use of organic additives alone. Organic dyes due to their electronic charge and hydrophobicity/hydrophilicity can be used to either increase or decrease the bio-adhesive properties of polymers when incorporated into such polymers. The incorporation of oligomer compounds into a wide range of different polymers that are not normally bio-adhesive may be used to increase the adherence of the polymer to tissue surfaces such as mucosal membranes.

As will be readily apparent to those skilled in the pertinent art from the present disclosure, various chemical groups and/or bio-adhesive materials may be incorporated in the adhesive layer 28 medium or matrix so as to improve the interaction or connection with the cell or tissue. For instance, a cationic surface-active agent may be utilized so as to create a positively charged adhesive layer. Also, an anionic surface-active agent can be utilized to create a negatively charged adhesive layer. Further, a nonionic surface-active agent may be used to create a neutral charged adhesive layer, or a zwitterionic surface-active agent may be used to create a variable charged adhesive layer.

According to another beneficial aspect of the present disclosure, a polymer adhesive or adhesive layer may be utilized that is pH sensitive, or that is composed of purely pH sensitive materials, or that is comprised of a mixture of pH sensitive materials, salt-sensitive, water-sensitive and/or bio-adhesive materials (e.g., polymer based). The pH-sensitive and salt sensitive materials can be blended with an inert water sensitive material, for example. By inert is meant a material that is not substantially affected by a change in pH or salt concentration in a triggering range. By altering the proportion of a pH-sensitive material to inert material the time lag subsequent to triggering and prior to release can advantageously be tailored. For example, an adhesive or adhesive layer 28 according to at least one aspect of the present disclosure can be formed so as to be stable in a solution or environment until the pH increases above a trigger pH, which causes the adhesive layer 28 to be activated so as to, for example, attract to a targeted cell or tissue. Likewise, in at least one other aspect of the present disclosure, the adhesive layer 28 can be formed to be stable in solutions and as the pH drops below a trigger pH the adhesive layer 28 is activated to attract to a targeted cell or tissue. In at least one further aspect of the present disclosure, a pH-sensitive trigger means can be used that may be capable of losing its adhesive quality or strength, such as to degrade or dissolve, following, for instance, a triggering by a solution of the desired pH, either above or below the trigger pH.

This reduction in adhesion quality or strength may, for example, allow for a selective release from a cell or tissue.

Exemplary pH-sensitive materials may include for example, without limitation, copolymers of acrylate polymers with amino substituents, acrylic acid esters, polyacrylamides, phthalate derivatives (i.e., compounds with covalently attached phthalate moleties) such as acid phthalates of carbohydrates, amylose acetate phthalate, cellulose acetate phthalate, other cellulose ester phthalates, cellulose ether phthalates, hydroxy propyl cellulose phthalate, hydroxypropyl ethylcellulose phthalate, hydroxypropyl methyl cellulose phthalate, methyl cellulose phthalate, polyvinyl acetate phthalate, polyvinyl acetate hydrogen phthalate, sodium cellulose acetate phthalate, starch acid phthalate, styrene-maleic acid dibutyl phthalate copolymer, styrene-maleic acid polyvinyl acetate phthalate copolymer, styrene and maleic acid copolymers, formalized gelatin, gluten, shellac, salol, keratin, keratin sandarac-tolu, ammoniated shellac, benzophenyl salicylate, cellulose acetate trimellitate, cellulose acetate blended with shellac, hydroxypropylmethyl cellulose acetate succinate, oxidized cellulose, polyacrylic acid derivatives such as acrylic acid and acrylic ester copolymers, methacrylic acid and esters thereof, vinyl acetate and crotonic acid copolymers.

Those skilled in the pertinent art, from the present disclosure, will readily appreciate variations and/or alternative adhesive materials and/or arrangements, suitable for use in association with a connecting interface having an adhesive layer, such as discussed herein, that may be made without departing from the spirit and scope of the present disclosure.

Referring generally to FIGS. 3-9, according to another illustrative aspect of the present disclosure, a connecting interface may have one or more micro-structures and/or nano-elements operatively associated therewith. For example, with particular reference to FIG. 3, there is shown a schematic representation of an electro-statically actuated (ESA) element 32 that may be operatively associated with a support 10 so as to form at least a portion of a connecting interface. As shown, element 32 may, for example, be a composite structure including a first level or element 34 (e.g., a connecting or engaging member) capable of selectively engaging or connecting to a targeted cell or tissue, a second level or element 36 (e.g., an actuator) operatively associated with the first element 34 so as to facilitate the selective actuation of first element 34, and a third level or element 38 (e.g., a connecting interface) capable of being connected to, integrated in, or otherwise associated with the support of the present disclosure.

Concerning the first element 34, according to an aspect of the present disclosure, such element may, for example, have two components, a first component 40 having certain predefined characteristics and a second component 42 also having certain predefined characteristics, at least some of which being distinct from those of the first component 40. For example, in an illustrative aspect of the present disclosure, the first component 40 is a polymer layer or film such as acrylate and the second component 42 is a conductive layer or film such as chromium.

Turning to the second element 36, according to another aspect of the present disclosure, such element may have one or more components. For example, in an illustrative aspect of the present disclosure, the second element 36 is an electrode 44 operatively associated with the first element 34 via a polymer (e.g., acrylate) film or layer 46 so as to facilitate a voltage difference being selectively applied between the electrode 44 and the second, preferably conductive, component 42 of the first element 34. With regard to the third element 38, such element, in accordance with still another aspect of the present disclosure, may be a substrate 48 (e.g., composite or other) suitable for cooperating with a support such as disclosed via the present disclosure.

Having identified and discussed some of the more pertinent aspects and features relative to the ESA element 32, attention is now given to how such element may be utilized as, or in association with, a connecting interface.

As shown via FIGS. 4 and 5, one or more ESA elements 32 may be operatively associated with a support 10 so that when ESA elements 32 are actuated (e.g., via a voltage difference applied between the first and second elements 34, 36), an electrostatic force draws or pulls the first element 34 thereof toward a corresponding second element 36, which element, as shown, may be arranged via the third element 38 and/or the support 10 so that each first element 34 is at least somewhat flush, and preferably substantially flush or flat, with the support surface 18 when actuated. Hence, in accordance with an aspect of the present disclosure, when the ESA element 32 is actuated, the first element 34 thereof preferably will not adhere to or engage a targeted cell or tissue 30 (shown via FIG. 4). Whereas, when the ESA element 32 is not actuated (e.g., no voltage difference applied between the first and second elements 34, 36), the electrostatic force is eliminated and the first element 34 thereof preferably withdraws or pulls away from the second element 36 so as to be capable of adhering to or engaging a targeted cell or tissue 30 (shown via FIG. 5).

Thus, according to a beneficial feature of the present disclosure, as the ESA element 32, which element may be made to be as small as a few hundreds of a micrometer or less, is capable of selectively connecting to a targeted cell or tissue via a controllable actuation means, the use of such elements in association with a support preferably capable of carrying and/or delivering different substances (e.g., medicaments, agents, and/or nanoparticles, whether smart or conventional) advantageously allows for a variety of different diagnostic, monitoring, and/or treatment processes or protocols to be more effectively and efficiently employed. For example, as shown via FIG. 6, according to an aspect of the present disclosure, the ESA element 32 via the first element 34 thereof may be operatively associated with the support 10 and a reservoir and/or a delivery means so as to facilitate, inter alia, substance delivery over an extended period of time and/or in higher local concentration. That is, the first element 34 of the ESA element 32 may serve not only as a means for selectively engaging or adhering to a targeted cell or tissue, but may in addition, or alternatively, serve as a means for selectively releasing or delivering any of a variety of substances 50 according to any of a variety of diagnostic, monitoring and/or treatment schedules or prescriptions.

Those skilled in the pertinent art, from the present disclosure, will readily appreciate variations and/or alternative materials and/or arrangements, suitable for use in association with, or in place of the ESA element 32, that may be used or made without departing from the spirit and scope of the present disclosure. For example, as the voltages necessary for effective actuation depend to large extent on the stiffness or thickness of the first element 34, the thickness of the first element can be adjusted so as to lower or otherwise modify the voltage required for effective actuation. That is, if, for instance, the first element 34 is made so as to be less than about 1 μm thick, as the actuation effect is accomplished via electrostatics, the current necessary for effective actuation is sufficiently small that the energy required may, in certain aspects of the disclosure, be provided via a battery sized so as to be accommodated directly by the support 10.

It will also be readily appreciated from the present disclosure that first element 34 may be shaped or configured so as to optimize adhesion or engagement with a targeted cell or tissue. For example, the thickness of the first element 34 may be varied so as to define gripping structures and/or the first element 34 may be provided with (e.g., a coating) an adhesive substance (e.g., lecithin or some other polymer/bio-adhesive such as discussed above) so as to improve adhesion upon engagement with a cell or tissue. In addition, propulsion means (e.g., a miniaturized jet pump) may be used to facilitate effective adhesion or engagement with a cell or tissue via, for example, providing an extra push to the first element 34 so that such element is anchored into the cell or tissue. Such propulsion means may also be used to facilitate disengagement and/or release from the cell or tissue.

It will further be readily appreciated from the present disclosure that any number of ESA elements 32 may be operatively associated with a support 10 so as to actuate individually, simultaneously, or sequentially according to any of a variety of combinations so as to facilitate engagement and/or disengagement relative to a cell or tissue. Also, as shown via FIG. 5, ESA elements 32 may be arranged to engage and/or disengage a cell or tissue from different perspectives or directions so as to be able to adjust to and/or accommodate for different environment conditions (e.g., fluid flow, surface topography, etc.)

It will still further be readily appreciated from the present disclosure that a variety of other means for actuation may be used in addition, or as an alternative, to electrostatics (e.g., light, pH, temperature, magnetism, etc.).

With particular reference now to FIG. 7, another example is shown via a schematic representation of a nano-pillar array 50 that may be operatively associated with a support 10 so as to form at least a portion of a connecting interface 16 in accordance with an illustrative aspect of the present disclosure. The nano-pillar array 50 can include any number of nano-pillars preferably based on Microelectromechanical Systems (MEMS), Nanoelectromechanical Systems” (NEMS), and/or other Micro- or Nano-technology. As shown, each nano-pillar 52, when activated, interacts with a targeted cell or tissue 30 via individual adjustment in response to contact with the cell or tissue surface 54. That is, once activated each nano-pillar 52 adjusts so as to correspond to the surface topography of the cell or tissue 30. Consequently, due primarily to the relatively conformed and close proximity contact between individual nano-pillars 52 and the surface 54, a relatively large contact surface results, which in turn leads to a relatively strong adhesion to the surface 54 due at least in large part to Van der Waals force, as well as an additional capillary force resulting from fluid commonly associated with cells or tissues filling up between adjacent nano-pillars 52. According to a beneficial aspect of the present disclosure, the size of each nano-pillar, the density or configuration of the nano-pillar array may be predefined so as to effectively adhere or engage a particular targeted cell or tissue 30. For example, a pillar size and array density can be determined based on known or measurable cell or tissue surface roughness, support weight and anticipated surface contact area between pillar array and targeted cell or tissue surface.

Those skilled in the pertinent art, from the present disclosure, will readily appreciate variations and/or alternative materials and/or arrangements, suitable for use in association with, or in place of the nano-pillar array 50, that may be used or made without departing from the spirit and scope of the present disclosure. For example, one or more nano-pillar 52, as with ESA element 32, can be provided with a coating of an adhesive substance (e.g., lecithin or some other polymer/bio-adhesive such as discussed above) so as to improve adhesion upon engagement with a cell or tissue. Also, each nano-pillar 52, according to an aspect of the present disclosure, can be individually actuated or actuated in conjunction with any number of other nano-pillars 52 via, for example, mechanical, electrical, electro-mechanical, chemical, electro-chemical, photo-chemical, or other like means suitable to selectively cause such nano-pillars 52 to change between at least two states (e.g., an engagable state and a disengagable state).

Turning to FIGS. 8 and 9, according to yet another illustrative aspect of the present disclosure, a connecting interface may have one or more suction elements 56 operatively associated therewith. For example, with particular reference to FIG. 8, there is shown a schematic representation of a suction system 58 suitable for use in association with a support 10 so as to form at least part of a connecting interface 16. As shown, system 58 may, for example, include one or more suction elements 56 capable of selectively engaging or connecting to a targeted cell or tissue, and a pump 60 operatively associated with at least one of the suction elements 56 so as to facilitate the selective actuation thereof, and/or an adjustable cavity or chamber 62 also, like pump 60, operatively associated with at least one of the suction elements 56 so as to facilitate the selective actuation of such elements, and communications means 64 for operatively connecting at least one suction element 56 to the pump 60 and/or a controller 61, the communications means 64 being also preferably capable of being connected to, integrated in, or otherwise associated with any one or more of the illustrative supports of the present disclosure.

Concerning suction element 56, according to an aspect of the present disclosure, such element may, for example, take the form of a flexible cup-shaped structure with an outer rim 66 suitable to contact a cell or tissue 30. The rim 66 can have and/or define a variety of different geometries. For example, the rim 66 can have a smooth or uniform pattern defining at least a substantially annular orifice. The rim 66 may also have an inconsistent or non-uniform pattern and define a non-annular orifice (e.g., ovular or non-curvilinear).

Turning to the pump 60, according to another aspect of the present disclosure, the pump 60 can take any of a variety of conventional forms, and is preferably sized and configured so as to correspond to that of the support 10 to which it is associated. For example, should the support 10 take the form of a capsule, the pump 60 can be a micro-pump 60 suitable to be accommodated by such capsule. Additionally, as shown via FIG. 9, should the support 10 take the form of an endoscope or like device, the pump 60 can be an external pump operatively connected to the support 10 via a connecting tube 68.

With respect to the chamber 62, according to an illustrative aspect of the present disclosure, the suction element 56 preferably cooperates with a cell or tissue 30 so as to selectively define the chamber 62. Moreover, according to a preferred aspect of the present disclosure, within the chamber 62 an under-pressure may be selectively created so as to keep the suction element 56 engaged with or connected to the cell or tissue 30. This under-pressure may be created in any of a variety of ways. For example, suction element 56 may cooperate with pump 60 via communications means 64 so as to selectively reduce the pressure in the chamber 62 by removing a fluid (e.g., a solution) from within the chamber 62. The fluid can be natural to the body, such as, the fluid commonly associated with cells or tissues, or the fluid can be artificially provided by, for example, pumping or injecting water (or other solution such as a physiological salt solution, e.g., a 0.9% NaCl solution) into the chamber 62. In an aspect of the present disclosure, the artificial fluid or solution is preferably used, at least in part, to prepare the cell or tissue 30 and/or to sterilize the suction element 56. In addition, the artificial fluid or solution may include any of a variety of substances, such as, for example, bio-markers, nano-particles, medicaments, or other substances for interacting with the cell or tissue. Once the artificial fluid or solution is provided to the chamber 62, by, for example, pump 60 (or other injecting means) in cooperation with at least a facet of communication means 64 (e.g., a fluid tube or channel network) adhesion to the cell or tissue 30 may be accomplished by then reverse pumping or ejecting at least some of the fluid from the chamber 62 so as to create an under-pressure.

With particular reference to FIG. 8, according to still another illustrative aspect of the present disclosure, communication means 64 may likewise serve to connect one or more suction elements 62 to the controller 61 so that the controller 61 can influence the suction elements 62 so as to facilitate selective adhesion thereof to a cell or tissue 30. For example, as shown in FIG. 8, one or more valves 63 may be operatively associated with a suction element 62, with the valves 63 in turn preferably being operatively connected to the controller 61 via at least a facet of communication means 64 (e.g., an electronic signaling network) such that the valves 63 may be selectively opened and/or closed according to any of a variety of criteria and in response to communications received from the controller 61 so as to, among other things, at least facilitate the selective adhesion of a suction element 62 to a cell or tissue 30.

Having identified and discussed via illustrative examples certain beneficial aspects and features associated with the present disclosure, and more particularly the connecting interface elaborated on herein, it will be readily understood by those skilled in the pertinent art from the present disclosure that there may be modifications and/or additions made to the connecting interface of the present disclosure without departing from the spirit and scope hereof. For example, one or more changeable wetting layers may be operatively associated with a support 10 so as to form at least a portion of a connecting interface 16.

Turning now to FIG. 10, there is shown a flow diagram illustrating a method 100 according to yet another aspect of the present disclosure. Although steps are shown in FIG. 6 in a particular arrangement for purposes of illustration, in other aspects of the present disclosure, the steps may be performed in a different order or in an overlapping manner. For example, in certain aspects of the present disclosure, step 120 can be completed after step 130, as in the case when a targeted cell or tissue is detected or treated by some means other than via the support of the present disclosure.

In step 110 one or more supports are delivered to one or more areas in a body. Any of a variety of methods for delivering the supports may be used including, for example, injection into the blood stream, injection into tissue, oral ingestion, and/or direct point delivery, among others. In a preferred aspect of the present disclosure, the support(s) can be in the form of an ingestible capsule such as, for example, shown via FIG. 1. Alternatively, or in addition, one or more of the supports may take the form of an endoscope or like device such as, for example, shown via FIG. 5.

In step 120, according to a preferred aspect of the present disclosure, the support(s) are connected with a targeted cell or tissue (e.g., cancerous cell or tissue) via an ergonomic connecting interface, such as, for example, a connecting interface utilizing at least any one or more of the connecting means of the present disclosure.

In step 130, at least one operation (e.g., medicament delivery) may be performed relative to a targeted cell or tissue. The operation(s) can be performed with or without support(s) being connected to the targeted cell or tissue. For example, according to one aspect of the present disclosure, a support or some other means may be utilized to accomplish or perform an operation of detecting or treating the targeted cell or tissue before the support connects to such cell or tissue. In another aspect of the present disclosure, once a support is connected to a targeted cell or tissue, such support may be utilized, for example, to deliver any of a variety of substances to the targeted cell or tissue. The substance delivery may be random, but is preferably controlled. As evidenced via the present disclosure, a connected support 10 preferably can improve medicament efficacy, reduce or eliminate causes relating to patient non-compliance to a prescription, help to maintain a steady medicament concentration, minimize the number of medicament administrations necessary, monitor a disease in real-time over a treatment course, and/or stabilize the interaction between a diagnostic and/or treatment tool or support and a cell or tissue so as to, among other things, beneficially allow for higher quality data or information to be obtained (e.g., via imaging).

If at least one operation is performed relative to a targeted cell or tissue, then via step 135, control may pass to at least one of steps 150, 160 or 170. If the at least one operation of step 130 has not been performed or completed relative to a targeted cell or tissue, then via step 135, control may pass to step 140.

In step 140, the at least one operation of step 130 may be continued or the support(s) may disconnect or release from the targeted cell or tissue, if applicable, so as to be able to move to a new area or exit the body. Relocating or moving the support(s) may be accomplished by any of a variety of known processes (e.g., self propulsion, peristalsis, etc.). In certain aspects of the present disclosure, the support(s) can be tracked or monitored via signal transmission (e.g., radio frequency (rf)) to a remote monitoring or observation unit.

In step 150, the support(s), upon performance of at least one operation relative to a targeted cell or tissue, may disconnect or release from such cell or tissue, if applicable, so as to be able to perform at least one other operation (e.g., image) relative to the targeted cell or tissue, or so as to be able to move to a new area or exit the body via step 140. It is noted that according to at least one aspect of the present disclosure, it may be that multiple supports are utilized to accomplish any of a variety of operations individually or in combination, and hence that at any one time, one or more supports may be connected to, or released from, a targeted cell or tissue.

In step 160, according to particular aspect of the present disclosure, at least one additional support is connected to the targeted cell or tissue so as to, for example, supplement or replace, the already connected support(s). Such additional support, in one aspect of the present disclosure, is preferably capable of accomplishing at least one additional operation. For example, and without limitation, in an illustrative aspect of the present disclosure, a first support may be used to deliver a medicament to a targeted cell or tissue, with a second support used to image or monitor any reaction or change in the targeted cell or tissue resulting from the medicament treatment.

In step 170, according to still another aspect of the present disclosure, the support(s) perform at least one additional operation relative the targeted cell or tissue. According to one aspect of the present disclosure, the additional operation may be performed while the support(s) remain connected to the targeted cell or tissue. Alternatively, in another aspect, the additional operation may be performed while the support(s) are disconnected or released from the targeted cell or tissue, such as discussed above relative to step 150. In certain aspects of the present disclosure, the additional operation may be identical or related to the operation previously performed, or it may be a variant thereof. In other aspects, the additional operation may be an entirely distinct operation separate and apart from the previous operation. For example, an operation of a first type can be performed so as to have a first effect on a targeted cell or tissue, while an operation of a second type can be performed (subsequently or contemporaneously) so as to have a second effect on the targeted cell or tissue. The effects provided by the different operations may be equivalent, identical, diverse, assorted, cumulative, cooperative, interactive, or otherwise related so as to accomplish any of a variety of different purposes (e.g., detecting, imaging, diagnosing, treating, etc.) with respect to the targeted cell or tissue.

As will be readily apparent from the present disclosure to those skilled in the pertinent art, variations to the foregoing method may be made without departing from the spirit and scope of the present disclosure. For example, certain steps may be accomplished via two or more supports such as discussed herein. In addition, with respect to step 170, for instance, in another aspect of the present disclosure, the additional operation can be performed by more than one support (e.g., an endoscope and an ingestible capsule). That is, a first support (e.g., an endoscope) may be used to guide or direct a second support (e.g., a capsule) to a targeted cell or tissue, while the second support accommodates and delivers at least one medicament to the targeted cell or tissue so as to have an affect thereon. Further, with respect to at least the steps identified and discussed above, according to yet another aspect of the present disclosure, a number such steps may be initiated and/or terminated in response to an event such as, for example, the performance of an operation, or the elapse of a predetermined time amount, or the exhaustion of a predetermined resource amount (e.g., substance or medicament), or the detection of an adverse reaction relative to the cell or tissue, or a signal provided or generated by a support (e.g., via a sensor) or a remote facility (e.g., a monitoring device or processor that can have means for user input or control).

As many aspects, features and advantages identified and described herein are apparent from the foregoing detailed discussion, it is intended by the appended claims to cover all such aspects, features and advantages that fall within the spirit and scope of the present disclosure. Further, since numerous modifications and changes will readily occur to those skilled in the art, it is not desired to limit the scope of the present disclosure to the exact construction and operation illustrated and described, and accordingly all suitable modifications and equivalents may be resorted to as falling within the present disclosures scope. Thus, the exemplary aspects and/or features described herein are merely illustrative and the present disclosure specifically encompasses alternative and/or modified aspects and/or features of that which has been disclosed.

1. A support comprising:
at least one ergonomic connecting interface for selectively connecting the support to a cell or tissue in a body;
at least one module suitable for accomplishing one or more operations relative to the cell or tissue; and
a controller operatively associated with an ergonomic connecting interface and/or a module.

2. The support of claim 1, wherein a module includes at least of one or more reservoirs suitable for retaining at least one substance, with at least one delivery mechanism associated therewith for selectively delivering one or more substances, one or more imagers suitable for imaging the cell or tissue, one or more sensors suitable for detecting various characteristics or pathology directly or indirectly associated with the cell or tissue, and one or more illuminators for providing light to or about the cell or tissue.

3. The support of claim 1, wherein the support is ingestible.

4. The support of claim 1, wherein the controller is operatively associated with the module so as to control the selective actuation thereof.

5. The support of claim 4, wherein the controller is operatively associated with the connecting interface so as to control the selective actuation thereof.

6. The support of claim 4, wherein the controller includes at least one sensor that operates, at least in part, to signal a controlled delivery of one or more medicaments.

7. The support of claim 4, wherein the controller includes a communicator for communicating with at least one remote facility.

8. The support of claim 5, wherein the controller includes a communicator for communicating with at least one remote facility.

9. The support of claim 5, further comprising a delivery mechanism for the controlled delivery of one or more substances.

10. The support of claim 7, wherein the remote facility includes a processing device for signaling the controlled delivery of one or more substances.

11. The support of claim 1, wherein the connecting interface includes at least one of (i) a microstructure and/or nano-element arrangement, (ii) an adhesive layer arrangement, and (iii) a suction system.

12. The support of claim 11, wherein the microstructure and/or nano-element arrangement includes an array of nano-pillars based on MEMS.

13. The support of claim 11, wherein the microstructure and/or nano-element arrangement includes one or more electro-statically actuated micro-hooks.

14. The support of claim 11, wherein an adhesive layer is pH activated.

15. The support of claim 11, wherein an adhesive layer changes from a first state to a second state when an electric field is applied thereto.

16. The support of claim 15, wherein the first state facilitates connecting to a cell or tissue in a body while the second state facilitates release therefrom.

17. The support of claim 11, wherein suction system includes one or more suction elements that cooperate with a targeted cell or tissue to define an adjustable chamber.

18. A system comprising:
at least one support suitable for being internalized into a body;
at least one ergonomic connecting interface operatively associated with each support;
at least one module operatively associated with each support, each module being suitable for accomplishing one or more operations relative to a cell or tissue in a body;
a controller operatively associated with at least one module; and
a remote facility operatively associated with the controller.

19. The system of claim 18, wherein a connecting interface includes at least one of (i) at least one nano-pillar array, (ii) at least one electro-statically actuated element, (iii) at least one polymer adhesive layer, and (iv) a suction system.

20. A method comprising the steps of:
introducing at least one support to one or more areas in a body;
connecting at least one support to a targeted cell or tissue of the body;
performing, via at least one support, at least one operation relative to the cell or tissue; and
disconnecting the at least one support in response to an event.

21. The method of claim 20, wherein the event includes at least one of: (i) performance of an operation, (ii) elapse of a predetermined time amount, (iii) exhaustion of a predetermined resource amount, (iv) detection of an adverse reaction relative to the cell or tissue, and (v) a signal provided internal to or external from a support, such signal being either automatically or manually generated via a sensor and/or a remote facility.
